(19) United States
(12) Patent Application Publication
(10) Pub. No.: US 2020/0025749 A1
(43) Pub. Date: Jan. 23, 2020

(54) DETECTION DEVICE AND DETECTION SYSTEM

(71) Applicant: Tamkang University, New Taipei City (TW)

(72) Inventors: Chih-Hsin Chen, New Taipei City (TW); Jhih-Wei Huang, New Taipei City (TW)

(73) Assignee: Tamkang University, New Taipei City (TW)

(21) Appl. No.: 16/424,218

(22) Filed: May 28, 2019

(30) Foreign Application Priority Data
Jul. 17, 2018 (TW) 107124703

(51) Int. Cl. G01N 33/53; G01N 21/21; B01L 3/00

(52) U.S. Cl. CPC G01N 33/5304; G01N 21/21; B01L 2300/0809; B01L 2300/168; B01L 2300/12; B01L 3/508

(57) ABSTRACT

A detection device includes a rectangular tube, an alignment film, a probe, and a nematic liquid crystal (LC). The rectangular tube has a tube wall and a receiving space located inside the tube wall. The tube wall has one or more light-transmittable region. The alignment film is located on the tube wall and corresponds to the light-transmittable region. The probe is distributed on the alignment film. The nematic LC is located in the receiving space. The probe includes an antibody, an antigen, or both the antibody and the antigen. Furthermore, a detection system is also provided.

### CROSS-REFERENCE TO RELATED APPLICATION

This non-provisional application claims priority under 35 U.S.C. § 119(a) to Patent Application No. 107124703 in Taiwan, R.O.C. on Jul. 17, 2018, the entire contents of which are hereby incorporated by reference.

### BACKGROUND

### Technical Field

The instant disclosure relates to an immunoassay detection technology, and in particular, to a detection device and a detection system for performing immunoassay by using liquid crystal (LC) molecules.

### Related Art

An LC-type detection system obtains an optical signal by using a double refraction property of an LC and then determines a detection result according to the optical signal. The system achieves many advantages, for example, marking is not required, the system is cheap and convenient to use, a result can be observed with naked eyes, the result is easy to interpret, a user can operate the system without specialized training, and the system can be designed to be a portable detection device. Such convenient detection systems mainly use three different detection methods: LC-solid interfaces, LC-liquid interfaces, and LC droplets.

An LC-solid interface system has always been a system most suitable for immunoassay among the LC-type detection systems. However, the LC-solid interface system is the most complex one among the three types of systems, and human errors also occur in the LC-solid interface system most easily. Generally, when the LC-solid interface system is operated, probe molecules first need to be modified on a glass surface in an array form, and then a solution to be tested is laid on the glass surface to react with the probe molecules. However, because the LC-type detection system has good sensitivity, due to subtle errors caused by various reasons (such as operating environment, component manufacturing, sample pretreatment, and different operators) during the operation process, the system may interpret the result mistakenly.

### SUMMARY

In an LC-solid interface system, a main error comes from component manufacturing. During manufacturing of components of an LC-solid interface, a film with a fixed thickness needs to be used for partitioning, so as to form a filling space required by an LC. However, a thickness difference of this filling space has obvious impact on an experiment result.

Accordingly, an embodiment of the instant disclosure provides a detection device, including a rectangular tube, an alignment film, a probe, and a nematic LC. The rectangular tube has a tube wall and a receiving space located in the tube wall. The tube wall has at least one light-transmittable region. The alignment film is located on the tube wall and corresponds to the light-transmittable region. The probe is distributed on the alignment film. The nematic LC is located in the receiving space. The probe includes at least one selected from a group consisting of an antibody and an antigen.

Further, another embodiment of the instant disclosure provides a detection system, including a detection device, a visible light source, two polarizers, and an optical sensor. The detection device includes a rectangular tube, an alignment film, a probe, and a nematic LC. The rectangular tube has a tube wall and a receiving space located in the tube wall. The tube wall has at least one light-transmittable region. The rectangular tube has a first side and a second side opposite to the first side. The alignment film is located on the tube wall and corresponds to the light-transmittable region. The probe is distributed on the alignment film. The nematic LC is located in the receiving space. The probe includes at least one selected from a group consisting of an antibody and an antigen. The visible light source is located on the first side. The polarizers are located on the first side and the second side, respectively, and polarizing directions of the two polarizers intersect with each other. The optical sensor is located on the second side. The visible light source emits visible light, and the visible light passes through the polarizers and the rectangular tube to illuminate the optical sensor.

In one or more embodiments, the probe has a concentration of 10 μg/mL to 20 μg/mL.

In one or more embodiments, the probe is a protein.

In one or more embodiments, a ratio of a tube width to a tube height of the rectangular tube ranges from 10 to 20.

In one or more embodiments, the rectangular tube includes two first plates disposed opposite to each other and two second plates disposed opposite to each other. Two sides of each first plate are connected to the two second plates, and two sides of each second plate are connected to the two first plates. A distance between the two first plates is the tube height of the rectangular tube, and a distance between the two second plates is the tube width of the rectangular tube. The at least one light-transmittable region is located on each first plate. More specifically, the at least one light-transmittable region is located at one end of each first plate. In other one or more embodiments, the at least one light-transmittable region is located on each first plate and each second plate.

Therefore, through the detection device and the detection system provided in one or more embodiments of the instant disclosure, a distance between inner walls of the rectangular tube is fixed, thereby reducing an error possibility during operations. Moreover, through a specific binding between the probe and a to-be-tested substance, an LC arrangement direction is changed, so that an optical property of the detection device is changed. Therefore, observation can be performed with naked eyes or an instrument, so as to detect the to-be-tested substance.

### BRIEF DESCRIPTION OF THE DRAWINGS

The disclosure will become more fully understood from the detailed description given herein below for illustration only, and thus not limitative of the disclosure, wherein:

FIG. 1 is a three-dimensional diagram of a first embodiment of a detection device according to the instant disclosure;

FIG. 2 is a first schematic sectional view of a first embodiment of a detection device according to the instant disclosure;

FIG. 3 is a second schematic sectional view of a first embodiment of a detection device according to the instant disclosure;

FIG. 4 is a schematic three-dimensional diagram of a second embodiment of a detection device according to the instant disclosure;

FIG. 5 is a schematic three-dimensional diagram of a third embodiment of a detection device according to the instant disclosure;

FIG. 6 is a three-dimensional exploded diagram of an exemplary embodiment of a detection system according to the instant disclosure;

FIG. 7 is a result diagram of optical signals generated by suctioning, through capillarity, anti-HSA (human serum albumin antibody) with different concentrations into rectangular capillaries containing HSA (human serum albumin) with a concentration of 20 μg/mL;

FIG. 8 is a result diagram of optical signals generated by suctioning, through capillarity, anti-BSA (bovine serum albumin antibody) with different concentrations into rectangular capillaries containing BSA (bovine serum albumin) with a concentration of 20 μg/mL;

FIG. 9(a) and FIG. 9(b) are result diagrams of optical signals generated by suctioning, through capillarity, anti-HSA and anti-H-IgG (human immunoglobulin G antibody) each with a concentration of 20 μg/mL into rectangular capillaries containing HSA with a concentration of 20 μg/mL;

FIG. 10 is a result diagram of a concentration of a to-be-tested substance in a liquid sample to be tested and a capillary height; and

FIG. 11 is a diagram of a relationship between a concentration of a to-be-tested substance in a sample to be tested and a capillary height.

### DETAILED DESCRIPTION

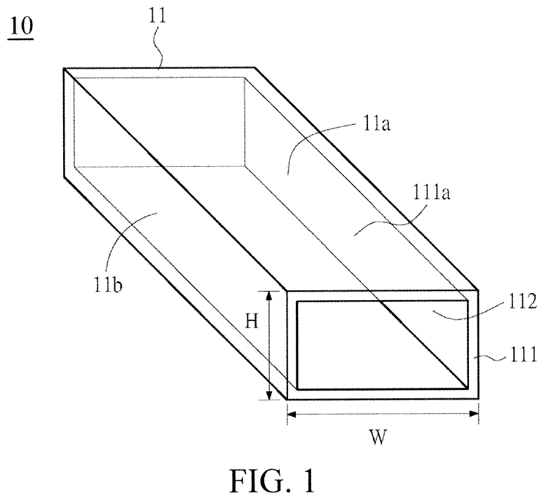

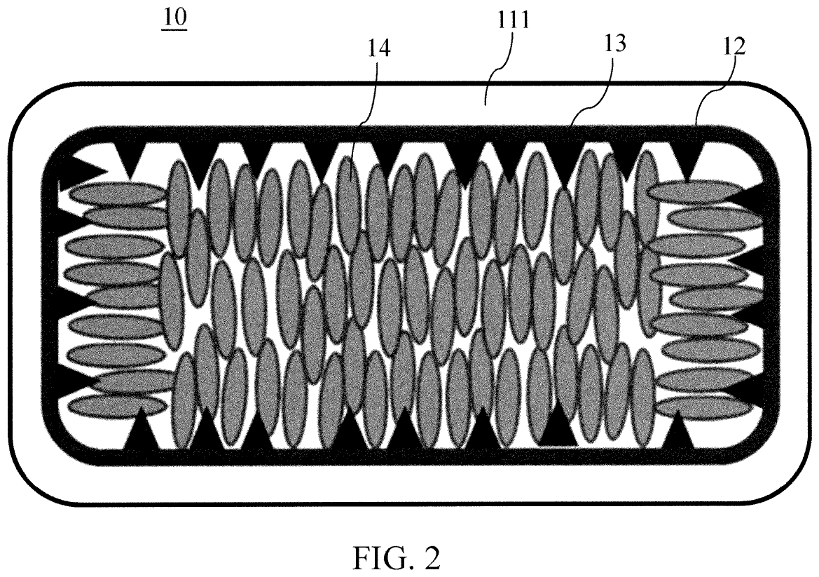

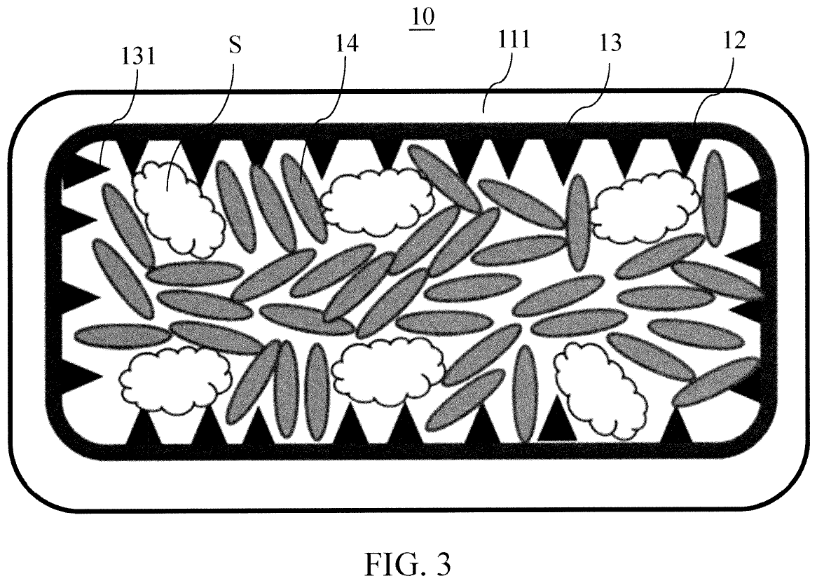

Referring to FIG. 1, FIG. 1 is a three-dimensional diagram of a first embodiment of a detection device according to the instant disclosure. Then, referring to FIG. 2 and FIG. 3 together, FIG. 2 is a first schematic sectional view of a first embodiment of a detection device according to the instant disclosure, and FIG. 3 is a second schematic sectional view of a first embodiment of a detection device according to the instant disclosure. As shown in FIG. 1 to FIG. 3, a detection device 10 includes a rectangular tube 11, an alignment film 12, a probe 13, and a nematic LC 14. In this embodiment, the detection device 10 may be applied to an immunoassay experiment for a liquid sample to be tested, and achieves a detection purpose through specific binding between an antibody and an antigen.

Referring to FIG. 1 to FIG. 3, the rectangular tube 11 has a tube wall 111 and a receiving space 112 located in the tube wall 111. The rectangular tube 11 is a capillary. The liquid sample to be tested and the nematic LC 14 are filled into the receiving space 112 through the capillarity. The tube wall 111 has at least one light-transmittable region 111a. In this embodiment, the light-transmittable region 111a can allow visible light to be passed therethrough. Specifically, in this embodiment, the rectangular tube 11 is made of a material (such as glass) that allows visible light to be passed therethrough. Therefore, the entire rectangular tube 11 is light-transmittable. In other words, in this embodiment, the rectangular tube 11 includes two first plates 11a disposed opposite to each other and two second plates 11b disposed opposite to each other. Two sides of each first plate 11a are connected to the two second plates 11b, and two sides of each second plate 11b are connected to the two first plates 11a. A distance between the two first plates 11a is a tube height H of the rectangular tube 11, and a distance between the two second plates 11b is a tube width W of the rectangular tube 11. The light-transmittable region 111a is located on each first plate 11a and each second plate 11b.

The alignment film 12, used for controlling LC arrangement, is located on the tube wall 111 and corresponds to the light-transmittable region 111a. A material of the alignment film 12 usually may be dimethyloctadecyl[3-(trimethoxysilyl)propyl] ammoniumchloride (DMOAP), octyltrichlorosilane (OTS), polyimide (PI), other alternative materials, or a combination thereof.

Further referring to FIG. 1 to FIG. 3, the probe 13 is distributed on the alignment film 12, and the probe 13 includes at least one selected from a group consisting of an antibody and an antigen. In other words, in one or more embodiments, the probe 13 includes an antibody (such as anti-HSA, human serum albumin antibody) and is used for detecting, in the liquid sample to be tested, an antigen (such as HSA, human serum albumin) having binding specificity with the antibody. Alternatively, in other one or more embodiments, the antibody includes an antigen (such as HSA) and is used for detecting, in the liquid sample to be tested, an antibody (such as anti-HSA) having binding specificity with the antigen. Alternatively, in other one or more embodiments, the probe includes both an antibody (such as anti-BSA, bovine serum albumin antibody) and an antigen (such as HSA) and is used for detecting, in the liquid sample to be tested, an antigen (such as BSA, bovine serum albumin) having binding specificity with the antibody and an antibody (such as anti-HSA) having binding specificity with the antigen. Therefore, different substances can be selected as the probe according to different experiment conditions.

Further referring to FIG. 1 to FIG. 3, the nematic LC 14 is located in the receiving space 112. Due to the presence of the alignment film 12, the nematic LC 14 is a regular arrangement. In this embodiment, the nematic LC 14 may be 4-cyano-4′-pentylbiphenyl (5CB), 4-cyano-4′-heptylbiphenyl (7CB), 4-cyano-4′-octylbiphenyl (8CB), 4-cyano-4′-oxyoctylbiphenyl, 4-cyano-4′-heptylterpheny, other alternative LCs, or a combination thereof.

Before the probe 13 is specifically bound with the to-be-tested substance S in the liquid sample to be tested, the nematic LC 14 is in an orderly arrangement (as shown in FIG. 2); when the probe 13 is specifically bound with the to-be-tested substance S in the liquid sample to be tested, the binding between the probe 13 and the to-be-tested substance S destroys the original orderly arrangement of the nematic LC 14 and also changes an optical signal (as shown in FIG. 3). Therefore, the to-be-tested substance S can be detected by using the probe 13 capable of being specifically bound with the to-be-tested substance S.

In one or more embodiments, the probe 13 is a protein. For example, the probe 13 may be the foregoing HSA, BSA, or the like. However, the probe 13 is not limited to the examples herein. Specifically, the probe 13 is bound, through an active region 131 thereof, with a corresponding region of the to-be-tested substance S.

In one or more embodiments, a ratio of the tube width W to the tube height H of the rectangular tube ranges from 10 to 20. If the ratio is excessively high, it may be difficult for the liquid sample to be tested and the nematic LC 14 to enter the receiving space 112 through capillarity. If the ratio is excessively low, the amount of the liquid sample to be tested and the nematic LC 14 that can enter the receiving space 112 may be small, therefore affecting the detection sensitivity. It should be noted that, the to-be-tested substance S can still be detected even if the ratio of the tube width W to the tube height H does not fall within the foregoing range. Therefore, the detection device according to one or more embodiments of the instant disclosure is not limited to the foregoing ratio range.

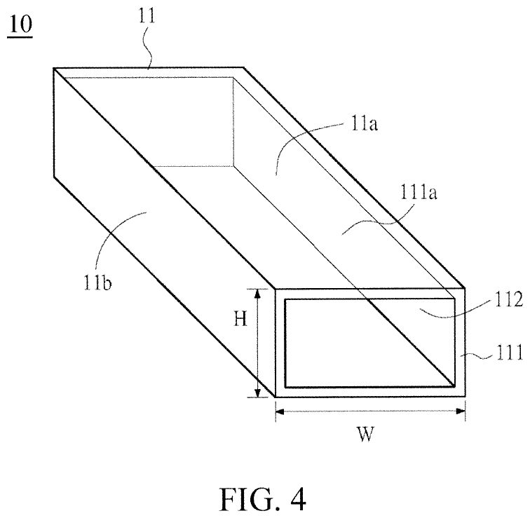

FIG. 4 is a schematic three-dimensional diagram of a second embodiment of a detection device according to the instant disclosure. Referring to 4, in this embodiment, the light-transmittable region 111a is located on each first plate 11a. It may be unnecessary to use a light-transmittable material for the whole rectangular tube, and therefore, the material cost can be reduced.

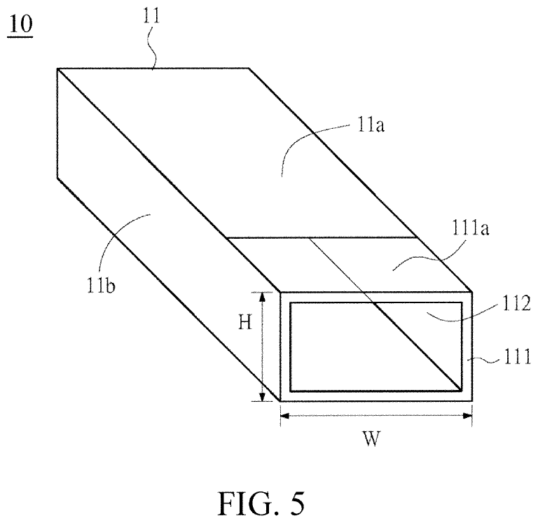

FIG. 5 is a schematic three-dimensional diagram of a third embodiment of a detection device according to the instant disclosure. Referring to FIG. 5, in this embodiment, the light-transmittable region 111a is further located at one end of each first plate 11a, so that the material cost can be further reduced. Herein, the position of the light-transmittable region 111a corresponds to a part, which is used for being in contact with the liquid sample to be tested, of the rectangular tube 11.

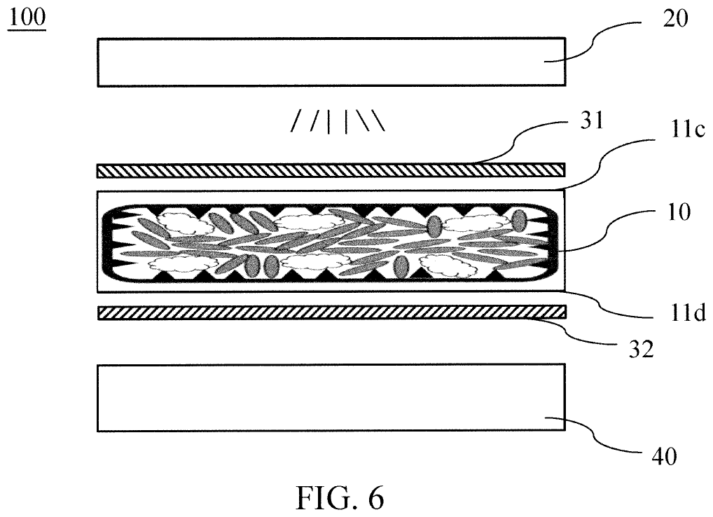

Referring to FIG. 6, FIG. 6 is a three-dimensional exploded diagram of an exemplary embodiment of a detection system according to the instant disclosure. Further refer to FIG. 1 to FIG. 3 together. As shown in FIG. 1 to FIG. 3 and FIG. 6, a detection system 100 includes a detection device 10, a visible light source 20, a first polarizer 31, a second polarizer 32, and an optical sensor 40.

It should be noted that, the detailed structure of the detection device 10 has been described in the preceding paragraphs. Therefore, only parts of the detection device 10 that are in coordination and association with other components in the detection system 100 are described herein. The rectangular tube 11 of the detection device 10 has a first side 11c and a second side 11d opposite to the first side 11c. The visible light source 20 is located on the first side 11c and can emit visible light. The first polarizer 31 and the second polarizer 32 are located on the first side 11c and the second side 11d respectively, and polarizing directions of the two polarizers 31 and 32 intersect with each other. Specifically, in an embodiment, the polarizing directions of the first polarizer 31 and the second polarizer 32 may be perpendicular to each other. In addition, an arrangement direction of the nematic LC 14 may be perpendicular or non-perpendicular (for example, parallel) to the two polarizers 31 and 32. The optical sensor 40 is located on the second side 11d. Therefore, when the visible light source 20 emits visible light, the visible light sequentially passes through the first polarizer 31, the rectangular tube 11, and the second polarizer 32, and then illuminates the optical sensor 40. Therefore, the optical sensor 40 can determine, according to a difference in optical signals, whether a to-be-tested substance S is detected. For example, the optical sensor 40 may simply record a brightness value displayed by the optical signal, or may be implemented by an image-capturing module.

Based on the foregoing description, when the light-transmittable region 111a is only configured on one end of the first plate 11a, an illumination range of the visible light source 20 and sizes of the polarizers 31 and 32 can be adjusted correspondingly. In other words, the material cost of the polarizers 31 and 32 can be reduced, and the quantity of light emitting components required in the visible light source 20 can be reduced.

Specifically, before the probe 13 is specifically bound with the to-be-tested substance S in the liquid sample to be tested, the nematic LC 14 is in an orderly arrangement. In this case, when incident light (that is, the visible light described above) passes through the first polarizer 31 and illuminates the nematic LC 14 in the detection device 10, the LC cannot change a polarization angle of the incident light, so that the direction of the light passing through the first polarizer 31 remains unchanged and the light cannot pass through the second polarizer 32. Therefore, the detection device 10 has low light transmittance, and obtains a dark signal. On the other hand, when the probe 13 is specifically bound with the to-be-tested substance S in the liquid sample to be tested, the original orderly arrangement of the nematic LC 14 is destroyed. In this case, the LC can hardly be orderly arranged, so that the direction of the light passing through the first polarizer 31 is changed, and a part of the light can pass through the second polarizer 32; therefore, the detection device 10 has high light transmittance.

For example, a method for manufacturing the detection device 10 may include the following process:

1. An alignment film preparation step:

A Decon-90 solution with a volume percentage concentration of 5% is suctioned into a rectangular capillary (that is, the rectangular tube) through capillarity, and stands for 12 hours. Then, the rectangular capillary is washed with deionized water through capillarity. Next, a DMOAP solution with a volume percentage concentration of 1% is suctioned into the rectangular capillary again through capillarity. Wait for 10 minutes. Finally, the rectangular capillary is washed with deionized water through capillarity, and is air-dried with nitrogen. The rectangular capillary is baked for 15 minutes in a vacuum oven at a temperature of 100° C., to obtain a rectangular capillary with DMOAP as an alignment film (which is referred to as a DMOAP rectangular capillary hereinafter).

2. A probe suction step:

A probe is suctioned into the DMOAP rectangular capillary through capillarity, so that the probe is distributed on the alignment film.

3. A step of suctioning a liquid sample to be tested:

The liquid sample to be tested is suctioned into the rectangular capillary having the probe and the alignment film again through capillarity.

4. An LC adding step:

LC molecules (5CB molecules are used herein) are suctioned into the rectangular capillary through capillarity.

It should be noted that, in the foregoing process, the liquid sample to be tested is suctioned first, and then the LC is added; however, the action sequence is not limited thereto.

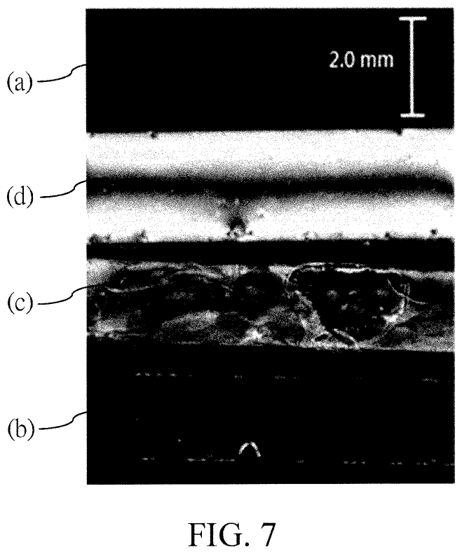

Referring to FIG. 7, anti-HSA with different concentrations (0 μg/mL in region (a), 2.5 μg/mL in region (b), 5 μg/mL in region (c), and 10 μg/mL in region (d)) are suctioned into rectangular capillaries containing HSA with a concentration of 20 μg/mL, respectively. After 20 minutes, non-specifically bound proteins are washed away by using tween-20, to prevent the proteins from interfering with the detection result. Finally, after air drying with nitrogen, the LC can be filled in to observe a detection result. In this embodiment, the used capillaries each have a width of 2 mm and a height of 0.1 mm. It can be learned from FIG. 7 that, a detection limit for anti-HSA is 5 μg/mL.

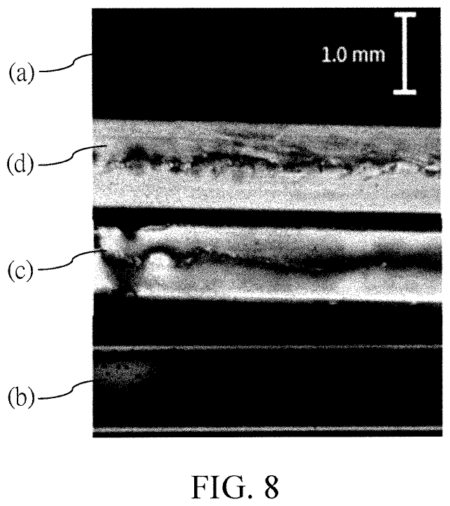

Referring to FIG. 8, anti-BSA with different concentrations (0 μg/mL in region (a), 2.5 μg/mL in region (b), 5 μg/mL in region (c), and 10 μg/mL in region (d)) are suctioned into rectangular capillaries containing BSA with a concentration of 20 μg/mL, respectively. After 20 minutes, non-specifically bound proteins are washed away by using tween-20, to prevent the proteins from interfering with the detection result. Finally, after air drying with nitrogen, the LC can be filled in to observe a detection result. In this embodiment, the used capillaries each have a width of 1 mm and a height of 0.1 mm. It can be learned from FIG. 8 that, a detection limit for anti-BSA is 5 μg/mL.

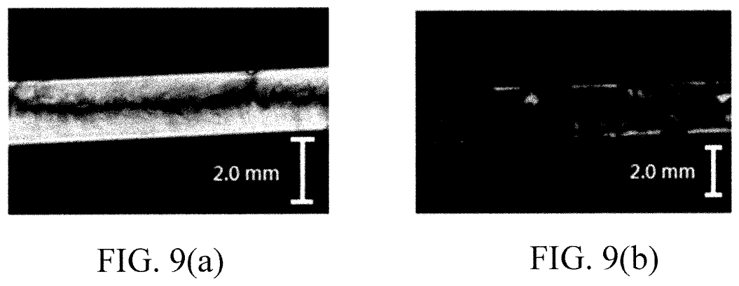

Referring to FIG. 9(a) and FIG. 9(b), anti-HSA and anti-H-IgG (human immunoglobulin G antibody) with the same concentration are suctioned into rectangular capillaries containing HSA with a concentration of 20 μg/mL (in FIG. 9(a), anti-HSA has a concentration of 10 μg/mL, and in FIG. 9(b), anti-H-IgG has a concentration of 10 μg/mL). After 20 minutes, non-specifically bound proteins are washed away by using tween-20, to prevent the proteins from interfering with the detection result. Finally, after air drying with nitrogen, the LC can be filled in to observe a detection result. In this embodiment, the used capillaries each have a width of 2 mm and a height of 0.1 mm. It can be learned from FIG. 9(a) and FIG. 9(b) that, when the liquid sample to be tested contains anti-H-IgG that does not have binding specificity with HSA, one can only observe a dark signal from the detection device. Therefore, the immunoassay has selectivity.

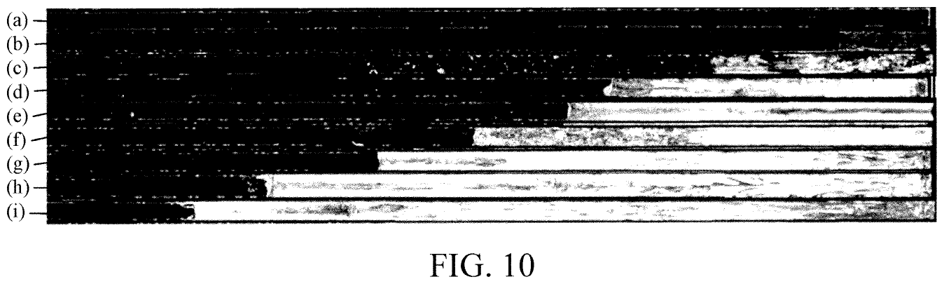

FIG. 10 is a result diagram of a concentration of a to-be-tested substance in a liquid sample to be tested and a capillary height. Anti-HSA with different concentrations (0 μg/mL in region (a), 1 μg/mL in region (b), 5 μg/mL in region (c), 10 μg/mL in region (d), 20 μg/mL in region (e), 30 μg/mL in region (f), 40 μg/mL in region (g), 50 μg/mL in region (h), and 60 μg/mL in region (i)) are suctioned into rectangular capillaries containing HSA with a concentration of 10 μg/mL through capillarity, respectively; therefore, there are different corresponding capillary heights for the rectangular capillaries. In this embodiment, the rectangular capillary shown in FIG. 1 is used, that is, the whole rectangular capillary is made of a light-transmittable material, to facilitate subsequent interpretation of the capillary height. Experiment results are shown in Table 1 below.

TABLE 1

| | Concentration (μg/mL) | | | | | | | | |
|---|---|---|---|---|---|---|---|---|---|
| | 0 | 1 | 5 | 10 | 20 | 30 | 40 | 50 | 60 |
| Capillary | 0 | 0.5 | 1 | 1.8 | 1.9 | 2.5 | 2.6 | 2.7 | 3.2 |
| length of | | | | | | | | | |
| Experiment | | | | | | | | | |
| 1 | | | | | | | | | |
| Capillary | 0 | 0.7 | 1.2 | 1.1 | 2 | 2 | 3 | 2.2 | 3 |
| length of | | | | | | | | | |
| Experiment | | | | | | | | | |
| 2 | | | | | | | | | |
| Capillary | 0 | 0.7 | 0.7 | 1.8 | 2.3 | 2.6 | 2.9 | 3.8 | 4.1 |
| length of | | | | | | | | | |
| Experiment | | | | | | | | | |
| 3 | | | | | | | | | |
| Average | 0 | 0.63 | 0.97 | 1.57 | 2.07 | 2.37 | 2.83 | 2.9 | 3.43 |
| capillary | | | | | | | | | |
| length | | | | | | | | | |

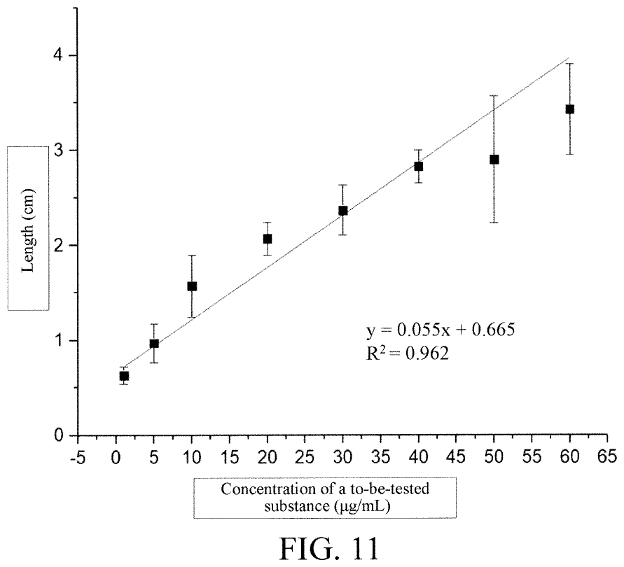

Based on the foregoing experiment results, a diagram of a relationship between the concentration of the to-be-tested substance in the sample to be tested and the capillary height is drawn, as shown in FIG. 11. As shown in the figure, within the range, the capillary height is in positive correlation with the concentration of the to-be-tested substance in the sample to be tested.

Therefore, through the detection device and the detection system provided in one or more embodiments of the instant disclosure, a distance between inner walls of the rectangular tube is fixed, thereby reducing an error possibility during operations. Moreover, through a specific binding between the probe and a to-be-tested substance, an LC arrangement direction is changed, so that an optical property of the detection device is changed. Therefore, observation can be performed with naked eyes or an instrument, so as to detect the to-be-tested substance.

What is claimed is:

1. A detection device, comprising:
a rectangular tube having a tube wall and a receiving space located in the tube wall, wherein the tube wall has at least one light-transmittable region, the rectangular tube comprises two first plates disposed opposite to each other and two second plates disposed opposite to each other, two sides of each first plate are connected to the two second plates, two sides of each second plate are connected to the two first plates, a distance between the two first plates is a tube height of the rectangular tube, and the at least one light-transmittable region is located on each first plate;
an alignment film located on the tube wall and corresponding to the at least one light-transmittable region;
a probe distributed on the alignment film; and
a nematic liquid crystal (LC) located in the receiving space;
wherein the probe comprises at least one selected from a group consisting of an antibody and an antigen.

2. The detection device according to claim 1, wherein the probe has a concentration of 10 μg/mL to 20 μg/mL.

3. The detection device according to claim 1, wherein the probe is a protein.

4. The detection device according to claim 1, wherein a ratio of a tube width to the tube height of the rectangular tube ranges from 10 to 20.

5. The detection device according to claim 1, wherein the at least one light-transmittable region is located at one end of each first plate.

6. A detection device, comprising:
a rectangular tube having a tube wall and a receiving space located in the tube wall, wherein the tube wall has a plurality of light-transmittable regions, the rectangular tube comprises two first plates disposed opposite to each other and two second plates disposed opposite to each other, two sides of each first plate are connected to the two second plates, two sides of each second plate are connected to the two first plates, and the light-transmittable regions are located on each first plate and each second plate;
an alignment film located on the tube wall and corresponding to the light-transmittable regions;
a probe distributed on the alignment film; and
a nematic liquid crystal (LC) located in the receiving space;
wherein the probe comprises at least one selected from a group consisting of an antibody and an antigen.

7. The detection device according to claim 6, wherein the probe is a protein, the probe has a concentration of 10 μg/mL to 20 μg/mL, and a ratio of a tube width to a tube height of the rectangular tube ranges from 10 to 20.

8. A detection system, comprising:
a detection device, comprising:
a rectangular tube having a tube wall and a receiving space located in the tube wall, wherein the tube wall has at least one light-transmittable region, the rectangular tube has a first side and a second side opposite to the first side, the rectangular tube comprises two first plates disposed opposite to each other and two second plates disposed opposite to each other, two sides of each first plate are connected to the two second plates, two sides of each second plate are connected to the two first plates, a distance between the two first plates is a tube height of the rectangular tube, and the at least one light-transmittable region is located on each first plate;
an alignment film located on the tube wall and corresponding to the at least one light-transmittable region;
a probe distributed on the alignment film; and
a nematic liquid crystal (LC) located in the receiving space;
wherein the probe comprises at least one selected from a group consisting of an antibody and an antigen;
a visible light source located on the first side;
two polarizers located on the first side and the second side, respectively, wherein polarizing directions of the two polarizers intersect with each other; and
an optical sensor located on the second side;
wherein the visible light source emits visible light, and the visible light passes through the polarizers and the rectangular tube to illuminate the optical sensor.

9. The detection system according to claim 8, wherein the probe has a concentration of 20 μg/mL.

10. The detection system according to claim 8, wherein the probe is a protein.

11. The detection system according to claim 8, wherein a ratio of a tube width to the tube height of the rectangular tube ranges from 10 to 20.

12. The detection system according to claim 8, wherein the at least one light-transmittable region is located at one end of each first plate.

13. A detection system, comprising:
a detection device, comprising:
a rectangular tube having a tube wall and a receiving space located in the tube wall, wherein the tube wall has a plurality of light-transmittable regions, the rectangular tube has a first side and a second side opposite to the first side, the rectangular tube comprises two first plates disposed opposite to each other and two second plates disposed opposite to each other, two sides of each first plate are connected to the two second plates, two sides of each second plate are connected to the two first plates, and the light-transmittable regions are located on each first plate and each second plate;
an alignment film located on the tube wall and corresponding to the light-transmittable regions;
a probe distributed on the alignment film; and
a nematic liquid crystal (LC) located in the receiving space;
wherein the probe comprises at least one selected from a group consisting of an antibody and an antigen;
a visible light source located on the first side;
two polarizers located on the first side and the second side, respectively, wherein polarizing directions of the two polarizers intersect with each other; and
an optical sensor located on the second side;
wherein the visible light source emits visible light, and the visible light passes through the polarizers and the rectangular tube to illuminate the optical sensor.

14. The detection system according to claim 13, wherein the probe is a protein, the probe has a concentration of 10 μg/mL to 20 μg/mL, and a ratio of a tube width to a tube height of the rectangular tube ranges from 10 to 20.
